(19) United States
(12) Patent Application Publication
(10) Pub. No.: US 2010/0323360 A1
(43) Pub. Date: Dec. 23, 2010

(54) Oligonucleotide arrangements, processes for their employment and their use

(76) Inventors: Thomas Ehben, Weisendorf (DE); Christian Zilch, Leipzig (DE)

Correspondence Address: HARNESS, DICKEY & PIERCE, P.L.C., P.O. BOX 8910, RESTON, VA, 20195, US

(21) Appl. No.: 12/805,878

(22) Filed: Aug. 23, 2010

Related U.S. Application Data
(62) Division of application No. 11/474,454, filed on Jun. 26, 2006, now abandoned.

(30) Foreign Application Priority Data
Jun. 27, 2005 (DE) 10 2005 029 811.7

(51) Int. Cl. C12Q 1/68; G01N 33/50

(52) U.S. Cl. 435/6; 436/94

(57) ABSTRACT

Oligonucleotide arrangements are disclosed which, in each case, have at least two oligonucleotide sequences linked via at least one spacer. A process is disclosed using the oligonucleotide arrangements for the amplification and/or detection of nucleic acid sequences with formation of crosslinked conglomerates. The process can be used, for example, for the sensitive, simple and inexpensive detection of nucleic acid sequences.

### PRIORITY STATEMENT

The present application is a divisional application of U.S. Ser. No. 11/474,454, filed on Jun. 26, 2006, which claims priority under 35 U.S.C. §119 on German patent application number DE 10 2005 029 811.7 filed Jun. 27, 2005, the contents of each of which are incorporated herein by reference in their entirety.

### FIELD

The invention generally relates to oligonucleotide arrangements, for example in each case containing at least two oligonucleotide sequences linked via at least one spacer (connecting piece) and/or to a process using the oligonucleotide arrangements for the amplification and/or detection of nucleic acid sequences for example, and/or their use in life science research and in high-throughput techniques.

### BACKGROUND

Nucleic acid assays are to an increasing extent an important instrument in order to obtain information about diseases, health risks and possibilities of treatment of a patient and are in particular suitable for the detection of pathogens, since they are able to identify pathogens specifically with the aid of certain DNA or RNA sequences occurring in these.

Compared to laboratory diagnostic methods used up to now, these tests offer many advantages, because the culturing of bacteria or viruses or the detection of an immune response in the human body is preparatively complicated and often necessitates uneconomically long analysis times.

Most conventional immunoassays for the diagnosis of infections can only detect the presence of pathogens indirectly via the determination of an immune response of the human body. Using a nucleic acid assay which analyzes the genome of the pathogen, information can be additionally obtained about the pathogen, e.g. about subtypes or mutations which have led to resistances to certain medicaments. Such information has additional therapeutic relevance. This specific pathogen information is used today, inter alia, in the diagnosis of HIV, HCV, Chlamydia and gonorrhea.

By the direct detection of the pathogens, nucleic acid assays can often detect infectious diseases in an earlier stage than conventional assays, if, for example, a virus is already present in the patient in latent form, but the disease has still not broken out and thus has still not induced an immune reaction in the patient.

Up to now, in use essentially two variants of a nucleic acid assay have contributed to the prior art.

(1) This is, on the one hand, the homogeneous nucleic acid assay using hybridization probes. In this assay, certain sequence sections which are contained in a nucleic acid-containing sample are hybridized with labeled oligonucleotides (probes) complementary to these sections and detected by way of the labels.

The polymerase chain reaction (PCR) can furthermore be part of a nucleic acid assay and replace or generate the above-mentioned hybridization probes. Here, free deoxynucleotides are added to starter oligonucleotide sequences (primers) utilizing the template effect of a target sequence which is present in a DNA sample, and with the aid of a DNA polymerase which replicates the target sequences to a great extent. These nucleic acid sequences thus obtained by amplification are then also designated as amplicons.

Alternative processes for the PCR or its further developments are, for example, strand displacement amplification (Walker, G. T., et al., Nucleic Acid Res. (1992) 7, 1691-1996), ligase chain reaction, rolling circle amplification, nucleic acid sequence-based amplification, branched DNA, transcription-mediated amplification, hybrid capture and Invader.

Customary known methods for detection include, for example, the employment of fluorescent labels, enzymes, radioisotopes, magnetic particles, quantum dots (nanocrystals), detection by means of antibodies and intercalating fluorescent dyes.

Using homogeneous nucleic acid assays, for detection, at the start molecules are as a rule added to the liquid phase which emit a fluorescent optical signal whose intensity is dependent on the course of the amplification reaction. The following processes are most frequent:
FRET (fluorescent resonant energy transfer): During each phase of the amplification cycles on which the nucleic acids are present in single-stranded form, fluorescent molecules and “quenchers” accumulate on this in immediate proximity. The quenchers lead by resonance effects to a local quenching of the optical emission of the fluorescent molecules as long as this proximity is maintained. If an amplification of the respective nucleic acid occurs, here both the fluorescent molecules and the quenchers are separated from the nucleic acid Sand lose their spatial proximity. The optical quenching breaks down and a fluorescent signal can be measured through the transparent reaction chamber.
Molecular beacon or hairpin: Molecules are added to the liquid phase which are complementary to the target sequence sought (or to a part of it). At two remote sites of such a beacon are situated one fluorescent and one quencher molecule each. These sites are connected loosely to one another by complementary groups. If a beacon is situated free in solution, it therefore shapes itself such that fluorescent molecule and quencher are spatially near together and the optical emission is quenched. As soon as a high concentration of the nucleic acid sought is present by amplification, the beacons accumulate on these nucleic acid molecules using a group complementary hereto. This takes place during the phases of the amplification in which the nucleic acids are present in single-stranded form. The loose complementary compounds originally existing are broken up in the course of this, the beacons are extended and fluorescent molecule and quencher are spatially separated from one another. Signal emission occurs.
Hybridization probes: Here, for example, two different fluorescent labels are present in the liquid phase, which only emit a suitable fluorescent optical signal in immediate spatial proximity to one another. In this process, one of the labels functions as an acceptor, the other as a donor. The emission is initiated by charge carrier exchange. Both labels are coupled using one hybridization probe in each case, which have a complementary sequence to regions of the target sequence sought lying close together. If a strong amplification of the target sequence and an increase in its concentration occur, the labels can accumulate on the target sequence in increased amount in immediate proximity to one another. As a result, a charge carrier exchange is made possible, and an optical signal is emitted.
Intercalating fluorescent dyes: These substances accumulate between the base pairs of double-stranded DNA, whereby signal emission is initiated. If the concentration of this double-stranded DNA increases as a result of amplification (in each case after each elongation phase of the cycles), the signal emission thus also increases.

These processes are established in research and in some cases in medical routine, but partly have the disadvantage that they involve a considerable outlay in terms of apparatus and the costs for the special labels are relatively high. These processes, however, can also be used in the detection step for at least one embodiment of the present invention.

In the technical realization of the nucleic acid assays for clinical routine, two variants are of importance.

In the case of homogeneous assays, the necessary chemical reactions take place in a homogeneous liquid phase. The nucleic acid obtained and prepared, for example, from blood or other patient samples is cyclically amplified here, i.e. in each reaction cycle controlled externally by temperature variations, the number of nucleic acid molecules (amplicons) increases provided the sequence sought was present in the patient sample.

Specific primer pairs in the solution in this case see to it that only the target sequence sought is amplified. By mixing various primer pairs, it is also possible to amplify a number of target sequences simultaneously (multiplex process).

Qualitative measurements are possible by checking, after a number of amplification cycles defined beforehand, whether the concentration of the doubled nucleic acid molecules exceeds a certain threshold value.

For quantification, this concentration is determined after each cycle and the number of cycles until a certain threshold value is achieved is determined. This number is a measure of the concentration of the sought nucleic acid in the patient sample.

The multiplex process is also employed here in order also to additionally increase controls, in parallel to the patient sample, which are added to the solution in known amount before the beginning of the amplification.

(2) As a further process of a nucleic acid assay, “microarrays” (Occasionally also called “gene chips or biochips”) are known. Here, the nucleic acid assays are carried out in the presence of a DNA sequence connected to a support material (like a capture molecule). Instead, however, of measuring the concentration of the sought target sequence in the homogeneous liquid phase, after the amplification a hybridization is carried out in which locally immobilized capture molecules specifically accumulate certain nucleic acids. The concentrations of the accumulations on the carrier material are determined metrologically as a result of the increased signal emission. In qualitative assays, the exceeding of a certain threshold value is an index of the presence of a sought target sequence in the patient sample. In quantitative assays, the amount of the nucleic acids in each case accumulated on specific capture molecules is determined. It is a measure of the concentration of the respective nucleic acid sequence in the patient sample.

An advantage of microarrays compared to homogeneous assays is the high parallelism. In the amplification, primers can be employed which in some cases are not specific for certain target sequences, but amplify certain sequence sections independently of genetic variations of the patient sample. During the hybridization, a fine differentiation then takes place by the use of a large number of different capture molecules. The microarrays developed for clinical diagnosis in some cases have over 100 different capture molecules.

In microarrays, during the amplification all amplified copies of the nucleic acid sequences are coupled to a label. Usually, this is a fluorescent optical label, e.g. Cy3 or Cy5. If a certain nucleic acid sequence is present in the patient sample in high concentration, it is strongly amplified and is accumulated during the hybridization by the respective capture molecules in high concentration. Locally increased fluorescent emission occurs, which is determined for the various capture molecules metrologically.

This process is also established, but has the disadvantage that the signal emission is adversely affected by the limited hybridization efficiency, i.e. each of the capture molecules does not also actually accumulate a labeled nucleic acid molecule. Additionally problematical is the necessity to differentiate the emission of the marked nucleic acids accumulated by capture molecules from those not accumulated, that is nucleic acids situated free in solution. This is achieved either by a number of washing steps after hybridization is terminated or by three-dimensional resolving signal detection (e.g. measurement in the evanescent field or confocal optics).

By way of the technology of amplification, the sensitivity can be greatly increased, which is especially important for the detection of nucleic acids which occur in only a very small concentration in the patient sample. For the determination of this concentration, a high sensitivity and a large dynamic bandwidth is very important.

In the amplification, a certain section on the target DNA of the material to be investigated (e.g. of a bacterium, virus or chromosome) is copied with the aid of suitable oligonucleotides as primers. The primers are customarily linked to suitable labels (having, for example, fluorescent, radioactive or enzymatic properties), which make possible detection after the preparation of the amplification products (Schweitzer, B., Kingsmore, S., Curr. Opin. Biotech. (2001) 12, 21-27).

In WO 03/038059 A2, oligonucleotide primers for PCR reactions are described which are bonded to nanoparticles, in particular colloidal gold particles. The primers are coupled to the gold particles via linkers, e.g. thiol groups or carbon chains.

In the Journal of the American Chemical Society (2002) 124, 7314-7323, Nicewarner-Pena et al have likewise described oligonucleotides bonded to nanoparticles for hybridization reactions and enzymatic primer extension.

In Langmuir (2004) 20, 10246-10251, DNA:nanosphere bioconjugates were described by Godrich et al, which form aggregates with complementary nucleic acids.

### SUMMARY

Although the abovementioned techniques have a distinct sensitivity, they include, however, the problems of a high expenditure of time and in terms of apparatus, high costs of the labels and possibly expensive protective devices as in the case of the radioisotopes. An increasing demand thus prevails for simpler and less expensive measuring methods which make possible a high sample throughput and have at least comparable analytical power. Furthermore, the expenditure in terms of personnel and apparatus for the analysis should be kept as low as possible in order to make possible a decentralized employment of the method. At the same time, however, it should not have any negative influence on the sensitive reaction kinetics of the nucleic acid amplification.

At least one embodiment of the invention thus resides, inter alia, in making available oligonucleotide arrangements which in each case contain at least two, preferably more than three, particularly preferably more than 100 and in particular more than 1000, hybridizable oligonucleotide sequences connected by one or more spacers, where at least one of the spacers can contain at least one label. The labels can be fluorescent molecules, other optically active molecules, magnetic particles, quantum dots, enzymes, electrically active molecules or radioisotopes.

Furthermore, the labels can, however, also act affinitively to their complementary partner, such as, for example, in antigen (hapten)/antibody interactions (e.g. digoxigenin or biotin) or thiol groups on gold surfaces. The labels can, however, also serve only for assisting the conglomerate formation. As such “passive” labels, inter alia, metals, metal ions and polymers can be employed. Markers which induce an optical color change as a result of the conglomerate formation are also part of at least one embodiment of the invention.

Finally, the detection of the networks formed can also be carried out purely optically, such as, for example, by way of turbidity measurement, or gravimetrically, such as, for example, by means of the piezosensor technique of Siemens AG.

Furthermore, at least one embodiment of the invention makes available a process for the amplification and/or detection of nucleic acids using these labels. The hybridizable oligonucleotide sequences are also described below as primers or primer sequences.

At least one embodiment of the invention is furthermore distinguished in that “upstream”- (complementary to the sense DNA) and “downstream”-(complementary to the antisense DNA) hybridizable oligonucleotide sequences can simultaneously be part of an oligonucleotide arrangement or the oligonucleotide arrangements in the total of in each case oligonucleotide arrangements having only “upstream”- and those having only “downstream”-hybridizable oligonucleotide sequences are combined.

In the oligonucleotide arrangements, the oligonucleotides serve as primers (starter oligonucleotides) in the usual manner for amplification reactions or as probes for the hybridization to give the oligonucleotides complementary to the target sequences.

The spacers disclosed in at least one embodiment of the invention, connecting the hybridizable oligonucleotides, are themselves not capable of hybridization and are composed, for example, of functionalized linear or branched carbon chains having, for example, 5 to 20 carbon atoms. Instead of carbon chains, the person skilled in the art, however, can also synthesize oligonucleotide arrangements which contain a different kind of spacer. A spacer can, according to the invention, simultaneously also bind more than two oligonucleotide sequences.

The oligonucleotide arrangements can furthermore be provided with at least one label, where this, in the case where only one spacer is present in the arrangement, is linked to the oligonucleotide arrangement, preferably in the region of the spacer. If the arrangement includes a number of spacers, the label is connected to at least one of these spacers.

The oligonucleotide arrangements can be employed in the microarrays or homogeneous assays described at the outset.

At least one embodiment of the invention furthermore relates to a process for the crosslinkage of nucleic acid sequences or molecules comprising such sequences in that the oligonucleotide arrangements according to at least one embodiment of the invention form conglomerates by coupling of the nucleic acid sequences to a number of the hybridizable oligonucleotide sequences of an oligonucleotide arrangement.

The nucleic acid sequences or molecules comprising such sequences are here preferably DNA substrands generated by an elongation in the course of an amplification reaction or of a primer extension.

The oligonucleotide arrangements assemble after the amplification, primer extension and/or hybridization reaction to give networks of nucleic acid sequences which measurably turbidify the reaction solution and whose concentration can thus be determined according to a further subject of the present invention by means of turbidity measurement or colorimetrically.

The low cost in terms of apparatus for detection is advantageous here, which according to at least one embodiment of the invention only extends to easily accessible spectrophotometers having a light source in the visible range. These light sources are, as a result, very simply maintained and can thus be employed, for example, in portable analysis apparatuses. Furthermore, the signal amplification leads to an improved signal-noise ratio as a result of conglomerate formation.

When employed in combination with homogeneous assays, no signal-emitting labels are necessary, as a result of which the costs per assay can be reduced and optionally even an assay evaluation using the naked eye is made possible.

When employed in combination with microarrays, as a result of the high number of label molecules accumulated in the area of the captors a particularly large concentration gradient occurs between labels on the surface and labels in solution. As a result, not only can any possible washing steps be omitted but also the requirements for a three-dimensional differentiation during the evaluation are reduced, e.g. the use of confocal optics. With minimization of the cavity volume (further increase in the concentration gradient) and dispensing with any washing steps, a three-dimensional resolution can thus be entirely dispensed with.

The oligonucleotide arrangements according to at least one embodiment of the invention thus surprisingly lead to an optimized signal emission and are thus a more sensitive, simpler and less expensive detection technique for amplified DNA sequences from nucleic acid assays.

At least one embodiment of the invention is furthermore characterized in that the primers comprise those molecules which hybridize with nucleic acids, such as, for example, DNA, RNA or derivatized nucleic acids and their mixtures.

At least one embodiment of the invention is also characterized in that the detection method can also be employed for “real-time” PCR and in reverse transcriptase PCR (RT-PCR) in the presence of reverse transcriptase for the determination of RNA.

Furthermore, the process according to at least one embodiment of the invention yields advantages in the area of the high-throughput process and in the mobile/decentralized employment area (‘point-of-care’ area).

At least one embodiment of the invention likewise relates to the use of this process in amplification reactions such as, for example, PCR, ligase chain reaction, strand displacement amplification, rolling circle amplification, nucleic acid sequence-based amplification, branched DNA, transcription-mediated amplification, hybrid capture or Invader.

A further subject of at least one embodiment of the invention is the employment of nucleic acid sequences linked to one another, which can be employed as hybridization probes (multivalent nucleic acid probes). These probes can now consist either of two or more nucleic acid sequences, which are complementary to a specific region or to different regions of the target sequence.

At least one embodiment of the invention furthermore relates to the fact that the probes mentioned can carry all label molecules known to the person skilled in the art.

The process according to at least one embodiment of the invention can be used in all fields in which nucleic acid analyses are operated, such as, for example, in medical, forensic, foodstuffs and environmental analysis, in plant protection, veterinary medicine or generally in life science research.

The detection process according to at least one embodiment of the invention can, for example, be advantageously employed in hereditary diseases and in oncology.

By way of example, the somatic genome can thus be investigated to see whether hereditary diseases are present (e.g. cystic fibrosis), whether a patient carries an increased disease risk (e.g. for breast cancer, detectable by mutations on the BRCA 1 and BRCA 2 genes) or whether a certain therapeutic is compatible with its individual genome (e.g. herceptin test of Abbott). A further field of use is HLA typing. In the case of tissue typing in the preliminary stages of transplants, nucleic acid assays allow significantly more sophisticated statements about the agreement of tissue types. This is especially important in bone marrow transplants, and better compatibilities can thus be achieved in organ transplants.

The steric influences often acting negatively on the sensitivity in conventional microarrays in the hybridization of the nucleic acid sequences on the immobilized capture molecules is encountered with the invention, because mainly the conglomerate formation leads to an improved signal-noise ratio and thus to a signal amplification.

An adjustment of the conglomerate formation rate can be achieved by variation of the following parameters:
1) The multivalence of the oligonucleotide arrangements, that is the number of primer sequences which an oligonucleotide arrangement contains.
2) The ratio of the “upstream” and “downstream” primer sequences in each case belonging to a target sequence, which are contained in an arrangement. An approximately balanced ratio makes possible a maximal hybridization of the amplicons complementary to one another, whereas a strongly “upstream”-or “downstream”-weighted ratio of the primers results in the number of amplicons produced, which can combine because of their complementarity, turning out to be lower and
3) optionally the addition of monovalent and lower valent primers for ‘dilution’ or increasing linearization of the networks.
4) A further control parameter is the extent of the presence of free primer.

According to a typical use of at least one embodiment of the invention, the necessary chemical reactions take place in a homogeneous liquid phase. The nucleic acid obtained and prepared, for example, from blood or other patient samples is added here to the reaction chamber, which already contains all necessary agents (including the oligonucleotide arrangements) and cyclically amplified, i.e. in each reaction cycle controlled externally by temperature variations the number of nucleic acid molecules increases exponentially, provided the sequence in question was present in the patient sample.

The specific primer sequences on the oligonucleotide arrangement ensure here that only the sought target sequence is amplified. By mixing of various oligonucleotide arrangements or of oligonucleotide arrangements having different primer sequences, it is also possible to amplify a number of target sequences simultaneously (multiplex).

Qualitative measurements are possible by testing after a number of reaction cycles defined beforehand whether the concentration of the accumulated nucleic acid molecules exceeds a certain threshold value. For a quantification, this concentration can be determined after each cycle and the number of cycles until a certain threshold value is achieved can be determined. This number is a measure of the concentration of the sought nucleic acid in the patient sample.

The multiplex process can be utilized here in order in parallel to the patient sample also to additionally amplify controls which are added to the solution in a known amount before beginning the amplification.

When using at least one embodiment of the invention in homogeneous assays, in each case a number of oligonucleotide sequences (primer sequences) which are specific for the target sequence are coupled to one another by suitable spacers. During the amplification cycles, the oligonucleotide arrangements are added by way of their primer sequences to the newly formed nucleic acid copies (amplicons) such that conglomerates of nucleic acid molecules result, whose size increases from cycle to cycle. The formation of these conglomerates depends strongly on the number of coupled primer sequences. From a certain number of cycles, the conglomerate size reaches dimensions which lead to an optical turbidification or a precipitation of the previously homogeneous liquid phase.

When illuminating the assay with visible light, this turbidification leads to scattering and/or absorption. This can be determined using a simple measuring technique known to the person skilled in the art and makes fluorescence optics superfluous. The assays become less expensive as a result, and the expenditure on apparatus falls. In the case of qualitative assays, even detection of the turbidification using the naked eye is conceivable, by means of which the expenditure on apparatus can be further reduced. This can be useful in decentralized and/or mobile applications having a low test throughput.

The action of the molecule conglomerates on the light transparency can be increased even further by additionally connecting the coupled primers to label molecules. These must not emit active signals, but can only be used during the amplification to amplify the turbidification by conglomerate formation. In addition to metals, suitable passive labels amplifying turbidification are also metal ions or polymers. Furthermore, in the presence of coloring substances a color deepening or a color change of the solution caused by conglomerate formation can be used for detection.

When employing the oligonucleotide arrangements according to at least one embodiment of the invention in customary microarrays having separate amplification and hybridization steps, networks of oligonucleotide arrangements comprising labels connected to one another via the amplicons are added to the immobilized capture molecules layerwise and these thus combine to give large conglomerates of nucleic acids and labels, preferably the network formation takes place layerwise growing from the carrier. Likewise, a subject of the invention can be that already extended, preformed conglomerates now add to the immobilized capture molecules, instead of individual, labeled oligonucleotide arrangements. Both possibilities, however, finally lead to an increase in the signal emission in the area of the respective capture molecules.

Although theoretically steric inhibition during the hybridization to the capture molecules can occur due to the size of the conglomerates, this aspect moves into the background if the low hybridization efficiency of a microarray is additionally taken into consideration, i.e. of the immobilized capture molecules, only a small part of nucleic acid molecules accumulates from the solution anyway. The order of magnitude of the conglomerate dimensions must orientate to the average spacing of this part of the capture molecules, in order that a significant steric inhibition is avoided.

The process according to at least one embodiment of the invention has the following advantages:
the signal amplification by conglomerate formation leads to an improved signal/noise ratio
on employment in combination with homogeneous assays, signal-emitting labels are not necessarily required, as a result of which the costs per assay can be reduced and optionally assay evaluation using the naked eye is made possible
on use in combination with microarrays, owing to the high number of the label molecules accumulated in the area of the immobilized captors, a particularly large concentration gradient occurs between labels on the surface and labels in solution. As a result, the requirements for possible washing steps or for a three-dimensional differentiation during analysis, e.g. the use of confocal optics, are decreased. Optionally, on minimizing the cavity volume (further increase in the concentration gradient) three-dimensional resolution can be dispensed with entirely.

At least one embodiment of the invention is also employable for carrying out in a novel assay, which is described in the simultaneously filed German Patent Application No. 10 2005 029 810.9 entitled “Processes for the detection of oligonucleotide sequences” of the same applicant and the same inventors, which corresponds to WO 2007/000408 A1, the entire contents of which are hereby incorporated herein by reference, is also made the subject of this application.

### BRIEF DESCRIPTION OF THE DRAWINGS

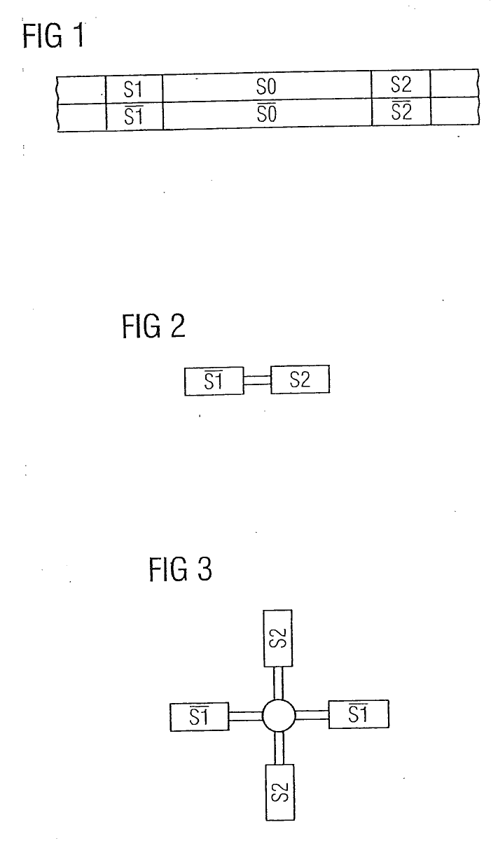

Example embodiments of the invention are illustrated, by way of example, in FIGS. 1 to 8:

### DETAILED DESCRIPTION OF THE EXAMPLE EMBODIMENTS

FIG. 1 shows: A double-stranded section of a DNA sequence comprising the target sequence, where S1 and S2 shorten the ends of the sense target DNA and S1* (shown in the figures as “S1 bar”) and S2* (shown in the figures as “S2 bar”) shorten the ends of the antisense target DNA. Accordingly, S1* and S2 are contained in the oligonucleotide arrangements as primers. S0 and S0* (shown in the figures as “S0 bar”) finally designate the DNA sequence included by S1/S2 and S1*/S2*. The actual target sequence S0 or S0* can in principle comprise the ends S1/S2 and S1*/S2*.

FIG. 2 shows: The minimal format of an oligonucleotide arrangement having two primers, which are connected via only one spacer.

FIG. 3 shows: The format of an oligonucleotide arrangement having, for example, four primers and alternatively one label, which is shown here by a centrally arranged circle.

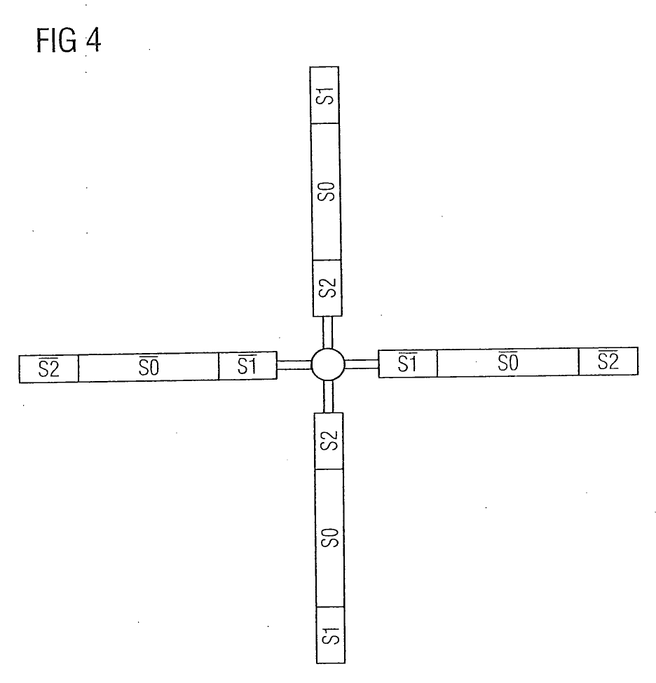

FIG. 4 shows: The individual oligonucleotide arrangement after an elongation reaction, such as, for example, amplification or primer extension, before conglomerate formation.

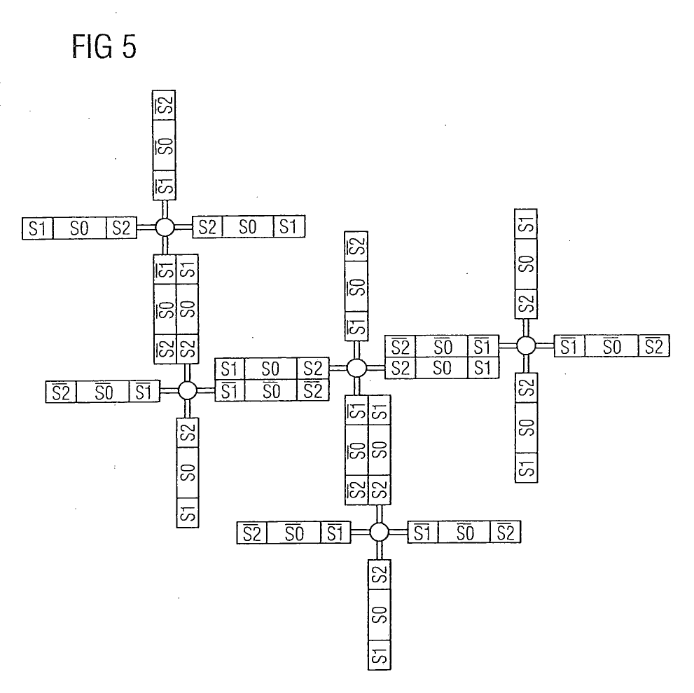

FIG. 5 shows: The schematic formation of molecule conglomerates or networks after hybridization of the arrangements of FIG. 4 has taken place.

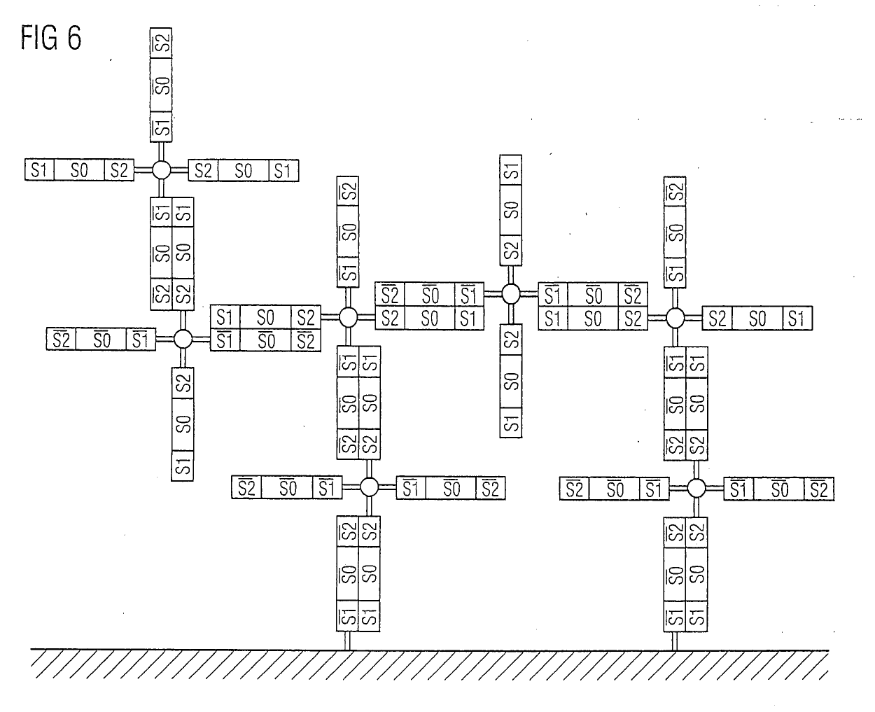

FIG. 6 shows: An arrangement, as can occur in microarrays, where one of the two primers was bound to a matrix as a capture primer, and the addition of the oligonucleotide arrangements according to the invention after amplification and hybridization has taken place in turn constructs a network which now, however, is present in immobilized form and connected to the carrier.

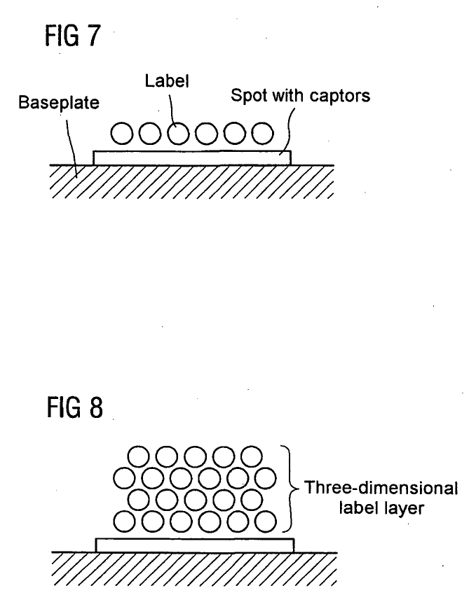

FIG. 7 shows: The two-dimensional arrangement, reduced to one dimension, of the label molecules coupled directly or indirectly to the amplicons after hybridization with the capture molecules has taken place, as exists in a known microarray.

FIG. 8 shows: The three-dimensional layerwise arrangement, reduced to two dimensions, of the label molecules coupled, for example, directly or indirectly to the amplicons after hybridization with the capture molecules has taken place, as occur in a microarray according to the invention. The layer-wise accumulation of the label molecules thus leads to an increase in the signal emission in the area of the respective capture molecules.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A process for determining target nucleic acids, comprising:
addition of a sample solution that includes a target nucleic acid to a reaction chamber including a plurality of agents comprising a plurality of oligonucleotide arrangements;
at least one of amplification, primer extension and reverse transcription;
hybridization; and
detection of the target nucleic acids,
wherein the oligonucleotide arrangements in each case contain at least two hybridizable oligonucleotide sequences linked by at least one spacer as primer sequences, the at least one spacer being selected from at least one of functionalized linear and branched carbon chains.

2. The process as claimed in claim 1, wherein the hybridization of amplicons resulting from the oligonucleotide arrangements and a target sequence with one another leads to conglomerate formation.

3. The process as claimed in claim 1, wherein the process is carried out in microarrays employing immobilized oligonucleotides, which in the course of the amplification form amplicons and bind these amplicons situated in solution.

4. The process as claimed in claim 1, wherein the process comprises, in the course of the amplification, hybridization of networks formed in solution to immobilized amplicons.

5. The process as claimed in claim 1, wherein detection comprises determining the presence of conglomerates qualitatively or a conglomerate concentration quantitatively by turbidity measurement, gravimetric and electrochemical methods, or, in the case of the presence of labels, by optical methods.

6. The process as claimed in claim 1, wherein detection includes determining a conglomerate concentration quantitatively by turbidity measurements or gravimetrically.

7. The process as claimed in claim 1, wherein the process is employed in the course of real-lime PCR and reverse transcriptase PCR in the presence of reverse transcriptase for determining RNA.

8. The process as claimed in claim 1, wherein the process is employed in a high-throughput process.

9. The process as claimed in claim 1, wherein amplification includes at least one of a polymerase chain reaction, a ligase chain reaction, strand displacement amplification, rolling circle amplification, a nucleic acid sequence-based amplification, a branched DNA, transcription-mediated amplification, hybrid capture and Invader.

10. The process as claimed in claim 1, further comprising employment of passive labels for accelerated conglomerate formation.

11. A method, comprising:
using the process as claimed in claim 1 in at least one of medical, forensic, foodstuff and environmental analysis, in plant protection, in veterinary medicine and in life science research.

12. A method, comprising:
using the process as claimed in claim 1 in high-throughput techniques and in a mobile and decentralized employment area.
